(19) United States
(12) Patent Application Publication
(10) Pub. No.: US 2003/0187269 A1
(43) Pub. Date: Oct. 2, 2003

(54) Novel process

(76) Inventors: Alan David Curzons, Arundel (GB); John Edward Richardson, Harlow (GB); Marian Wladyslaw Wood-Kaczmar, Sawbridgeworth (GB)

Correspondence Address: GLAXOSMITHKLINE, Corporate Intellectual Property - UW2220, P.O. Box 1539, King of Prussia, PA, 19406-0939, US

(21) Appl. No.: 10/140,845

(22) Filed: May 8, 2002

Related U.S. Application Data
(63) Continuation-in-part of application No. 09/676,330, filed on Sep. 29, 2000, now abandoned.
(63) Continuation-in-part of application No. 09/678,934, filed on Oct. 4, 2000, now abandoned.
(63) Continuation-in-part of application No. 09/678,548, filed on Oct. 5, 2000, now abandoned.
(63) Continuation-in-part of application No. 09/679,153, filed on Oct. 5, 2000, now abandoned.
Application No. 09/679,153 is a continuation of application No. 09/293,290, filed on Apr. 16, 1999, now abandoned.
Application No. 09/679,153 is a continuation of application No. 09/293,612, filed on Apr. 16, 1999, now abandoned.
Application No. 09/679,153 is a continuation of application No. 09/293,289, filed on Apr. 16, 1999, now abandoned.
Application No. 09/679,153 is a continuation of application No. 09/293,748, filed on Apr. 16, 1999, now abandoned.

(30) Foreign Application Priority Data
Apr. 17, 1998 (GB) 9808212.6
Apr. 17, 1998 (GB) 9808213.4
Apr. 17, 1998 (GB) 9808214.2
Apr. 17, 1998 (GB) 9808216.7

(51) Int. Cl. C07D 211/20; C07D 211/34

(52) U.S. Cl. 546/236; 546/238

(57) ABSTRACT

A process for the preparation of a compound of formula (1):

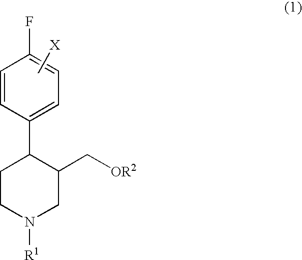

in which R¹ is an alkyl, arylalkyl, allyl, alkyloxycarbonyl, arylalkyloxycarbonyl, acyl or alkynyl group; and R² is substituted phenyl, especially 3,4-methylenedioxyphenyl; and X is hydrogen or a readily removable group, such as chlorine, bromine or iodine.

The present invention relates to a new process for preparing pharmaceutically active compounds and intermediates therefor.

Pharmaceutical products with antidepressant and anti-Parkinson properties are described in U.S. Pat. No. 3,912,743 and U.S. Pat. No. 4,007,196. An especially important compound among those disclosed is paroxetine, the (−) trans isomer of 4-(4′-fluorophenyl)-3-(3′,4′-methylenedioxy-phenoxymethyl)-piperidine. This compound is used in therapy as the hydrochloride salt to treat inter alia depression, obsessive compulsive disorder (OCD) and panic.

Previously published processes to paroxetine utilise as a key intermediate the carbinol

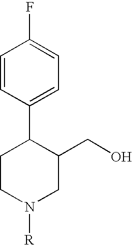

in which the piperidine nitrogen is protected by a group R, usually an alkyl (typically methyl) group. The N-substituted piperidine must be coupled with sesamol to make an N-substituted paroxetine analogue which is converted to paroxetine by removal of the nitrogen protecting group. The published coupling with the alcohol proceeds via an unisolated sulphonate ester intermediate.

In U.S. Pat. No. 3,912,743, Example 1, a solution of 3-hydroxymethyl-1-methyl-4-phenyl piperidine in pyridine is reacted with methanesulphonyl chloride. The pyridine is removed and the crude resultant sulphonate ester is treated with sodium methoxide and 4-methoxyphenol in methanol under reflux. In Example 5 of EP 0 152 273, 4-(4-fluorophenyl)-3-hydroxymethyl-1-methyl pyridine is dissolved in toluene together with triethylamine and cooled. Benzenesulphonyl chloride is added to this mixture. The resultant solution of the benzenesulphonic ester is then mixed with sodium methoxide and 4-methoxyphenol in methyl isobutyl carbinol and heated.

The present invention is based on the discovery of reaction conditions which are more suitable for industrial scale production.

In its broadest sense, the present invention provides a process for the preparation of a compound of formula (1):

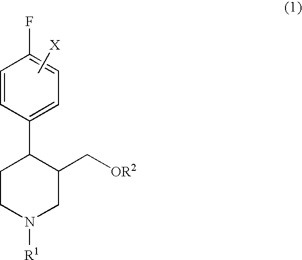

in which R¹ is an alkyl, arylalkyl, allyl, alkyloxycarbonyl, arylalkyloxycarbonyl, acyl or alkynyl group; R² is substituted phenyl, especially 3,4-methylenedioxyphenyl; and X is hydrogen or a readily removable group, such as chlorine, bromine or iodine.

The method comprises the steps of: (i) treating a solution of a compound of formula (2)

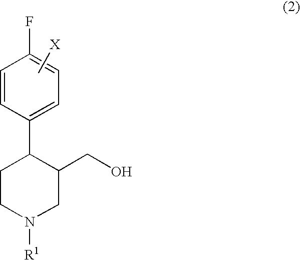

in which R¹ and X are as defined above, in dichloromethane with an excess of a sulphonyl chloride of formula R³SO₂Cl (wherein R³ is Ph, CF₃, CH₃, CH₂Ph, CH₂COPh, C₆H₄-4-MeO, C₆H₂-2,4,6-Me₃, C₆H₄-4-Me, CH₂Ph, or CH₂C₆H₄-4-Me) in the presence of a base; (ii) quenching the reaction mixture with an aqueous quench; (iii) separating the organic solution and removing the dichloromethane therefrom to obtain a sulphonate ester of formula (3)

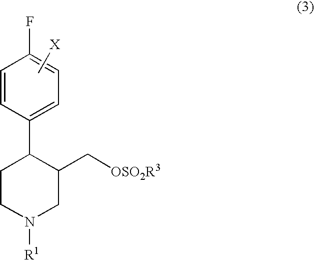

wherein R¹, R³ and X are as defined above; and (iv) coupling the sulphonate ester with a phenol of formula R²OH, especially sesamol (3,4-methylenedioxyphenol), in the presence of a base and in a solvent, wherein the solvent is dimethylformamide.

R³ is preferably phenyl, methyl or tolyl; more preferably phenyl.

Hitherto, the use of dichloromethane as the solvent for step (i) has been dismissed for several reasons including the relative insolubility of the intermediate sulphonate ester in dichloromethane resulting in premature crystallisation interfering with the washing stages. Careful balance of the reaction conditions has been found to be necessary. In particular, a relatively large volume of dichloromethane is preferably used, typically between 7 and 12 volumes, more preferably between 8 and 9 volumes. If insufficient solvent is used, components of the reaction may crystallise out of solution affecting the reaction rate and completeness in an unpredictable manner. Too much solvent results in poor yields, and in the context of an industrial process, volume inefficiency. The use of dichloromethane has also been dismissed in the synthesis of paroxetine because it reacts with sesamolate used in the subsequent stage (iv) to give a methylene bridged sesamol dimer and so must be completely removed.

Preferably the step (i) base is an amine, such as triethylamine, trimethylamine, diethylmethylamine or dimethylethylamine. More preferably, the amine is dimethylethylamine.

Accordingly, in a preferred process, a compound of formula (2) is dissolved in dichloromethane together with N,N-dimethylethylamine and the mixture is cooled, preferably to between −10° C. and +5° C. Benzenesulphonyl chloride in dichloromethane is added to the mixture with stirring. In this solution, the ratio by volume of sulphonyl chloride to dichloromethane is preferably between 3:1 to 1:5, more preferably about 1:1. It has also been determined that the molar ratios of amine to sulphonyl chloride to 3-hydroxypiperidine of formula (2) are preferably in the range 1.3-1.6:1.1-1.3:1, more preferably about 1.38:1.15:1. It has been observed that outside these ranges unpredictable problems can arise, in particular during the subsequent processing of the reaction mixture. In particular, it has been found that outside this range, the product sulphonate ester may fail to solidify, resulting in unacceptable levels of dichloromethane remaining and interfering with a subsequent coupling reaction of the sulphonate ester with an alcohol or phenol, such as sesamol. In particular, an excess of dimethylethylamine may give a toffee-like impure product upon work-up, but if too little is used the water quench may become inefficient at removing impurities and by-products, leading to batch failure.

The dimethylethylamine catalysed reaction of 4-(4-fluorophenyl)-3-hydroxymethyl-1-methylpiperidine with benzenesulphonyl chloride is so strongly exothermic that the benzenesulphonyl chloride must be added slowly to keep the reaction under control. It has been found that, far from resulting in a build up of unreacted reagents, cooling the reaction mixture allows for a more controlled reaction and reduces the addition time by around one half. Preferably, the cooling and rate of addition is adjusted to maintain a reaction temperature in the range of −10 to +5° C., more preferably between 0 and +2° C. Higher temperatures may cause excessive colour and impurities, whilst lower temperatures may result in incomplete reaction and low yields.

The step of a water quench has not been proposed previously and provides a quick and efficient method of purification. Conventionally, in the published processes, substantially only one equivalent of sulphonyl chloride is used, an excess being necessary only to make up for low assay or interfering side-reactions. In the case of the preferred range noted above, the excess of benzenesulphonyl chloride is real. Whilst an excess of benzenesulphonyl chloride within the ranges noted above provides an improved process to the sulphonate ester, the presence of chloride in a subsequent coupling with a phenol such as sesamol is disadvantageous as the sulphonyl chloride reacts with the sesamol, reducing the yield, but also can lead, in an unpredictable manner, to complete batch failure. The use of an aqueous quench overcomes this problem.

Preferably, the water of the quench includes an amine or inorganic base such as sodium hydroxide or sodium carbonate. Aqueous ammonia can be used, but it is preferred that water is used in combination with the amine already used in the reaction mixture. Preferential results have been obtained at the water quenching stage if the amine is dimethylethylamine rather than triethylamine. Unexpectedly, the sulphonyl ester has been found to be acceptably stable to water at ambient temperatures, although only within a narrow window. The pH of the solution is preferably controlled to be between about 6 and about 9, optimally between about 7.5 and about 8. Nevertheless, the procedure should be carried out reasonably quickly to avoid degradation of the product ester.

A processing advantage is obtained by adding the reaction mixture to the quenching water rather than the more conventional method of adding water to the batch. This allows deposited impurities to be removed by decantation. Furthermore, in the industrial context, it avoids the need for a water inlet pipe to the reaction vessel, failure of the isolating valve of which would lead to complete batch loss and also constitute a serious operating hazard. There is also no risk of contamination of subsequent batches by water in the reaction vessel.

Following the water quench, the organic phase is dried, preferably over magnesium sulphate, filtered, separated and evaporated to dryness. The evaporation conditions are important. It is important that the temperature is not allowed to rise above 35° C., since above this temperature, degradation may occur which, if excessive, can prevent crystallisation and cause the reaction to fail. It has been observed that there is less degradation when dichloromethane is the solvent than when other solvents are used

It has been determined that the use of dimethylformamide as the solvent for the step (iv) coupling with the phenol provides an advantageous work-up procedure as will be described below.

Preferably, the sulphonate ester of formula (3) is added to the solvent in a weight ratio of between about 1:4 and 1:12, more preferably between about 1:5 and 1:6, and the phenol R²OH is added to this solution.

Preferably the step (iv) base is an alkali metal salt such as carbonate, hydroxide or alkoxide. More preferably, the base is a sodium alkoxide, most preferably, sodium methoxide or ethoxide.

Preferably, the process uses between about 1.1 and 2 equivalents of base, more preferably between about 1.4 and 1.6 and most preferably about 1.5 equivalents of base.

Preferably, the base is added to a mixture containing the sulphonate ester of formula (2) and the phenol of formula R²OH in more than one portion, preferably in three portions. Preferably, the base is divided into three portions with a ratio of about 3:4:2.

Preferably, water is added to the reaction mixture prior to the addition of the last portion of base. Suitably, about 0.4 to 0.5 equivalents of water are used.

Improved results have been obtained with careful temperature control. Preferably, the initial addition of base is made at about 20° C. and the temperature is allowed to rise to about 45° C. during the reaction. For example, in a preferred embodiment, 0.5 equivalents of sodium methoxide are added to a mixture of the compound of formula (2) and the phenol R²OH in dimethylformamide at a temperature of between about 20-30° C. The mixture is stirred for about 10 minutes and a second portion of 0.67 equivalents of sodium methoxide is added at 30-45° C. and stirred for 1 hour. Water (0.4 equivalents) is added followed by a third portion of 0.33 equivalents of sodium methoxide. The mixture is then stirred at about 40-50° C. for a further 1-4 hours.

In an alternative embodiment, the sodium derivative of the R²OH phenol is pre-prepared and added to a solution of the sulphonate ester of formula (2).

Phase transfer agents may be used as desired to increase the solubility. As described above, the preferred solvent is dimethylformamide. Use of this solvent enables the product ether to be simply brought out of solution by addition of water. However, the conditions need to be controlled. If the DMF solution is allowed to cool before addition of water, the product is obtained as an oil, which may solidify, but entraining impurities. Water should be added to a warm DMF solution which is then allowed to cool, whereupon a purer crystalline product is obtained, which can be isolated by filtration, washing with water and drying under vacuum. For example, the reaction mixture, following addition of all the base, is warmed to about 50° C. and a total of about 2 volumes of water is added over the course of 2-6, typically about 4, hours maintaining the temperature at or around this level. The mixture is allowed to cool to around 20° C., whereupon the product ether begins to crystallise out of solution.

The process of this invention may be used to prepare active compounds described in U.S. Pat. No. 3,912,743 and U.S. Pat. No. 4,007,196, and preferably to prepare paroxetine.

Paroxetine is preferably obtained as or converted to the hydrochloride salt and most preferably the hemihydrate of that salt, as described in EP-A-0223403. The present invention includes within its scope the compound paroxetine, particularly paroxetine hydrochloride, especially as the hemihydrate, when obtained via any aspect of this invention, and any novel intermediates resulting from the described procedures.

Paroxetine is the (−)-trans isomer of 4-(4′-fluorophenyl)-3-(3′,4′-methylenedioxy-phenoxymethyl)-piperidine. Following the procedure of EP-0 152 273, optical resolution may be carried out prior to coupling with the phenol. Alternatively, resolution may be carried out at other stages, such as after deprotection of the piperidine nitrogen

Paroxetine obtained using this invention may be formulated for therapy in the dosage forms described in EP-A-0223403 or WO96/24595, either as solid formulations or as solutions for oral or parenteral use.

Therapeutic uses of paroxetine, especially paroxetine hydrochloride, obtained using this invention include treatment of: alcoholism, anxiety, depression, obsessive compulsive disorder, panic disorder, chronic pain, obesity, senile dementia, migraine, bulimia, anorexia, social phobia, pre-menstrual syndrome (PMS), adolescent depression, trichotillomania, dysthymia, and substance abuse, referred to below as “the Disorders”.

Accordingly, the present invention also provides:

a pharmaceutical composition for treatment or prophylaxis of the Disorders comprising paroxetine or paroxetine hydrochloride obtained using the process of this invention and a pharmaceutically acceptable carrier,

the use of paroxetine or paroxetine hydrochloride obtained using the process of this invention to manufacture a medicament for the treatment or prophylaxis of the Disorders; and

a method of treating the Disorders which comprises administering an effective or prophylactic amount of paroxetine or paroxetine hydrochloride obtained using the process of this invention to a person suffering from one or more of the disorders.

This invention is illustrated by the following Examples.

### EXAMPLE 1

Preparation of 4-(4′-fluorophenyl)-1-methyl-3-(3′,4′-methylenedioxy-phenoxymethyl) piperidine

Dichloromethane (1900 L) is charged to a dry vessel and further dried by azeotropic distillation. (3S,4R)-trans-4-(4-fluorophenyl)-1-methyl-3-hydroxymethylpiperidine (250 kg) is added with stirring, followed by dimethylethylamine (168 L), and the resulting mixture is cooled to 0° C. A solution of benzenesulphonyl chloride (164 L) in dichloromethane (250 L) is cooled to between −10 and 0° C., and then added to the 4-(4-fluorophenyl)-1-methyl-3-hydroxymethylpiperidine solution over approximately one hour, with continuous stirring and maintaining the temperature below 0° C. 30 minutes after the completion of the addition, at a temperature of 0-5° C., water (750 L) is added and the mixture stirred for 1 hour. The dichloromethane layer is then separated from the aqueous layer and dried with anhydrous magnesium sulphate (25 kg). The clear dichloromethane solution is isolated by filtration and the solvent removed by distillation at reduced pressure to leave a crystalline solid product (3S,4R)-trans-4-(4-fluorophenyl)-1-methyl-3-phenylsulphonyloxymethylpiperidine. Residual dichloromethane level <0.7%.

The solid product is charged to a vessel containing N,N′-dimethylformamide (1392 L) at 15-25° C. and stirred to dissolve. Sesamol (162 kg) is charged to the same vessel with continuous stirring. A first portion of sodium methoxide (30.2 kg) is added and the temperature maintained at 20-30° C.; after 10 minutes, a second portion (40.3 kg) is added and the mixture stirred for one hour, keeping the temperature within the range 30 to 45° C. Water is then added (8 kg), followed by a third portion of sodium methoxide (20.1 kg), and the mixture stirred for another hour at between 40 and 45° C. The temperature of the reaction mixture is then brought up to 50° C. and water (2 volumes) is added slowly over a period of 4 hours maintaining the temperature at 50° C. The temperature is then gradually brought down to 20° C. (2 hours) to complete the crystallisation, and the mixture stirred at that temperature for 1 hour. The product is then filtered, washed with water, and dried under vacuum at 40-60° C.

### EXAMPLE 2

Preparation of (3S,4R)-trans-4-(4-fluorophenyl)-1-methyl-3-methylsulphonyloxymethylpiperidine

(3S,4R)-trans-4-(4-fluorophenyl)-1-methyl-3-hydroxymethylpiperidine (20 kg) is dissolved in dichloromethane (150 L), treated with dimethylethylamine (13 L), and the resulting mixture cooled to 0° C. A solution of methanesulphonyl chloride (11 L) in dichloromethane (20 L) is cooled to between −10 and 0° C., and then added to the 4-(4-fluorophenyl)-1-methyl-3-hydroxymethylpiperidine solution over approximately two hours, with continuous stirring below 0° C. After the reaction is complete (30 minutes), the mixture is treated with a cold solution of aqueous ammonia (0.1 molar, 60 L) and stirred for 1 hour. The dichloromethane layer is then separated, dried with anhydrous magnesium sulphate (2 kg), filtered and evaporated to give a crystalline solid, substantially free of dichloromethane.

### EXAMPLE 3

Preparation of (3S,4R)-trans-4-(4-fluorophenyl)-1-methyl-3-p-toluenesulphonyloxymethyl-piperidine

(3S,4R)-trans-4-(4-fluorophenyl)-1-methyl-3-hydroxymethylpiperidine (10 kg) is dissolved in dichloromethane (50 L), treated with dimethylethylamine (6.5 L), and the resulting mixture cooled to 0° C. A solution of p-toluenesulphonyl chloride (9.8 kg) in dichloromethane (35 L) is cooled to 0° C., and added slowly (1 hour) to the stirred 4-(4-fluorophenyl)-1-methyl-3-hydroxymethylpiperidine solution, keeping the temperature below 5° C. After a further 1 hour stirring, the mixture is treated with cold aqueous sodium hydroxide (0.1 molar, 30 L) and stirred for 1 hour. The dichloromethane layer is then separated, dried with anhydrous magnesium sulphate (1 kg), filtered and evaporated to give a crystalline solid. Residual dichloromethane <1%.

1. A process for the preparation of a compound of formula (1): 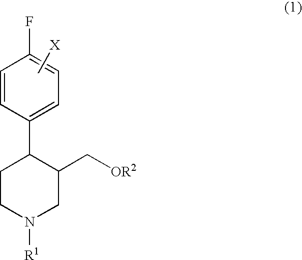
in which R¹ is an alkyl, arylalkyl, allyl, alkyloxycarbonyl, arylalkyloxycarbonyl, acyl or alkynyl group; and R² is substituted phenyl, especially 3,4-methylenedioxyphenyl; and X is hydrogen or a readily removable group, such as chlorine, bromine or iodine; which process comprises the steps of (i) treatment of a dichloromethane solution of a compound of formula (2) 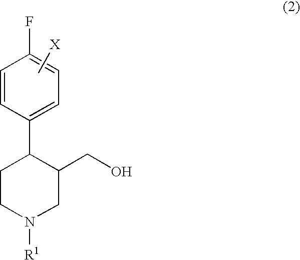
in which R¹ and X are as defined above, with an excess of a sulphonyl chloride of formula R³SO₂Cl; wherein R³ is Ph, CF₃, CH₃, CH₂Ph, CH₂COPh, C₆H₄-4-MeO, C₆H₂-2,4,6-Me₃, C₆H4-4-Me, CH₂Ph, or CH₂C₆H₄-4-Me; in the presence of a first base; (ii) quenching the reaction mixture with an aqueous quench; (iii) separating the organic solution and removing the dichloromethane therefrom to obtain a sulphonate ester of formula (3) 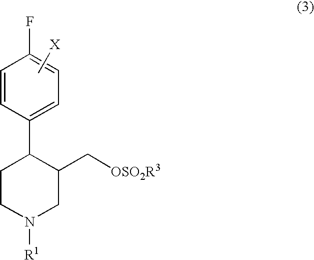
wherein R¹, R³ and X are as defined above; and (iv) coupling the sulphonate ester with a phenol of formula R²OH, especially sesamol (3,4-methylenedioxyphenol), in the presence of a second base and in a solvent, wherein the solvent is dimethylformamidine.

2. A method for the preparation of a compound of formula (1) as claimed in claim 1, in which about 7 to about 12 volumes of dichloromethane are used as the solvent in step (i).

3. A method as claimed in claim 1 wherein the first base is an amine, preferably dimethylethylamine.

4. A process as claimed in claim 1 wherein the R³ is phenyl, methyl, or tolyl, preferably phenyl.

5. A process as claimed in claim 1 in which the ratio of first base to sulphonyl chloride to piperidine of formula (2) is in the range 1.3-1.6:1.1-1.3:1.

6. A process as claimed in claim 5 in which the ratio is about 1.38:1.15:1.

7. A process as claimed in claim 1 in which the reaction temperature during addition of the sulphonyl chloride is maintained in the range of about −10 to about +5° C., preferably between about 0 and 2° C.

8. A process as claimed in claim 1 wherein the aqueous quenching water includes an amine or an inorganic base such as sodium hydroxide or sodium carbonate.

9. A process as claimed in claim 8 wherein the first base is an amine and also acts as the amine for the aqueous quench.

10. A process as claimed in claim 8 in which the pH of the aqueous quench is controlled to be between about 6 and about 9, preferably between about 7.5 and 8.

11. A process as claimed in claim 1 wherein the second base is an alkali metal alkoxide or hydroxide.

12. A process as claimed in claim 11 wherein the second base is a sodium alkoxide, preferably methoxide or ethoxide.

13. A process as claimed in claim 1 wherein between about 1.1 and 2 equivalents of the second base are used and the second base is added to a solution containing the sulphonate ester of formula (2) and the phenol.

14. A process as claimed in claim 13 in which between about 1.4 and 1.6 equivalents of second base are used, preferably about 1.5 equivalents.

15. A process as claimed in claim 13 in which the second base is added to the sulphonate ester and phenol solution in more than one portion.

16. A process as claimed in claim 15 wherein the base is added in three portions.

17. A process as claimed in claim 16 wherein the base is divided into three portions in the ratios of about 3:4:2.

18. A process as claimed in claim 15 wherein water is added to the reaction mixture prior to the addition of the final portion of base.

19. A process as claimed in claim 1 wherein between about 0.4 to about 0.5 equivalents of water are added.

20. A process as claimed in claim 2 wherein the compound of formula (1) is obtained from solution by allowing the temperature of the reaction mixture to warm during addition of the second base, with supplementary heating if necessary, and adding water to the warmed solution at such a rate as to substantially maintain the temperature of the solution and thereafter allowing the solution to cool, whereupon the compound precipitates and is removed by filtration.

21. A process as claimed in claim 20 wherein the temperature of the solution is allowed to reach or is maintained at about 50° C. during addition of the water.

22. A process as claimed in claim 1 wherein R² is 3,4-methylenedioxyphenyl, X is hydrogen and R¹ is methyl.

23. A process for preparation of (−)trans-4-(4′-fluorophenyl)-3-(3′,4′-methylenedioxy-phenoxymethyl)-piperidine that incorporates the process of claim 1.

24. A process according to claim 1 wherein, in the quenching step, the reaction mixture is added to the quench water.

25. (−)trans-4-(4′-fluorophenyl)-3-(3′,4′-methylenedioxy-phenoxymethyl)-piperidine whenever obtained by the process of claim 1.

26. (−)trans-4-(4′-fluorophenyl)-3-(3′,4′-methylenedioxy-phenoxymethyl)-piperidine as claimed in claim 25, in the form of a pharmaceutically acceptable salt such as the hydrochloride salt.

27. A pharmaceutical composition for treatment or prophylaxis of the disorders comprising a compound as claimed in claim 25 and a pharmaceutically acceptable carrier.

28. A method of treating the disorders which comprises administering an effective or prophylactic amount of a compound as claimed in claim 25 to a person suffering from one or more of the disorders.

29. A substantially pure compound of formula (1) as defined in claim 1 wherein R¹ and R² are as defined in claim 1 and X is H.

30. A compound as claimed in claim 29 wherein R¹ is methyl.

31. A compound as claimed in claim 29 wherein R² is methyl, phenyl or tolyl.

32. Substantially pure 4-(4-fluorophenyl)-1-methyl-3-phenylsulphonyloxymethyl-piperidine.
